(19) United States
(12) Patent Application Publication
(10) Pub. No.: US 2002/0138065 A1
(43) Pub. Date: Sep. 26, 2002

(54) Adult incontinence article that can be donned without removing clothing

(76) Inventors: Stacy Jean Yeater, Alpharotes, GA (US); Bernadette M. Gibbs, Statham, GA (US)

Correspondence Address: Hunton & Williams, 1900 K Street NW, Suite 1200, Washingiton, DC, 20096-1109, US

(21) Appl. No.: 09/814,767

(22) Filed: Mar. 23, 2001

(51) Int. Cl. A61F 13/15; A61F 13/20

(52) U.S. Cl. 604/395; 604/385.21; 604/385.23; 604/385.27; 604/385.29; 604/385.3; 604/391; 604/392; 604/396

(57) ABSTRACT

The garment of the present invention has a waist band with a crotch component that can be permanently attached to the back of the waist band and releasably attached to the front of the waist band. The crotch fits between the user's legs during use and the waist band wraps around the user's waist. The garment may have a one-, two-, or three-piece construction. The garment also has an absorbent core disposed generally in the crotch region. At least part of the interior surfaces of the garment are liquid-pervious to allow fluids to be absorbed into the absorbent core. The exterior surfaces of the garment are generally liquid-impervious, to prevent the fluid in the absorbent core from being released from the garment. The garment of the present invention may be donned quickly, easily, and with a minimum amount of exposure to unsanitary conditions or embarrassment. The garment of the present invention may also be donned by persons with limited use of their hands.

### FIELD OF THE INVENTION

The present invention relates generally to absorbent garments. Particularly, it relates to adult incontinence garments which may be donned without completely removing the user's clothing from the user's lower body.

### BACKGROUND OF THE INVENTION

Traditionally, disposable absorbent garments, such as training pants and adult incontinence products, were designed with fixed seams and permanent leg and waist holes, herein referred to as “unitary” construction. Other traditional disposable absorbent garments were designed with a pair of resealable side closure flaps that connected the rear half of the garment to the front half by stretching them around each side of the user's waist. This construction is referred to herein as “side-closure” construction.

Unitary absorbent garments typically could not be donned until the wearer had completely disrobed. The term “completely disrobed,” as used herein, refers to removing all clothing from the user's waist down. An example of a unitary article is disclosed in U.S. Pat. No. 4,205,679, issued to Repke et al.

Donning such garments in public places, such as public restrooms, can be difficult or embarrassing. It can be difficult to disrobe completely in a bathroom stall, privy, or other confined space. Furthermore, disrobing in such a place may expose the user's garments to unsanitary conditions, such as unclean floors or toilets. In order to find a larger or more sanitary place to don a traditional absorbent garment, the user may have to disrobe in a more open and public place, which can be embarrassing.

Side-closure absorbent garments typically may be donned while the user is partially disrobed. As used herein, “partially disrobed” refers to removing the user's clothing from the user's waist, but leaving the removed clothing around the legs of the user. U.S. Pat. No. 5,522,809, issued to Larsonneur, teaches a side-closure article that is demonstrative of this type of garment.

In order to properly align a side-closure garment, the user must correctly position the two side flaps in relation to the crotch. When properly aligned, the side flaps and crotch fit snugly and symmetrically around the wearer. Failure to properly align the garment can lead to discomfort and body exudate leakage. In addition to being time-consuming, positioning the side flaps generally requires two hands, which makes it particularly difficult to don the garment when the user is in a confined space and is also attempting to prevent his or her clothing from contacting unsanitary surfaces, such as the floor or a toilet. Furthermore, it is more difficult to align the garment when the user does not have access to a mirror. Side-closure systems also are subject to accidental opening by catching or snagging on the wearer's clothing.

The prior art has attempted to solve these problems by providing absorbent articles that have fixed waist bands and replaceable absorbent crotch elements that are attached to the waist band at the front and rear of the body. For example, U.S. Pat. No. 5,549,593, issued to Ygge et al., discloses a continuous elastic waist band with a series of fasteners around its perimeter, to which a replaceable absorbent crotch pad is attached. The absorbent article is connected to the waist band at both the front and the rear of the garment by means of a refastenable connection, such as hook and look fasteners.

To don the article disclosed in Ygge et al., the back portion must be attached first, the assembly then is rotated around the user's waist, and finally the front portion is attached. Such a procedure generally requires two hands, and can be especially difficult when the user is partially disrobed and attempting, at the same time, to prevent his or her outer clothing or body to come into contact with unsanitary surfaces. Also, both the front and rear connections must be properly aligned in order to ensure comfort and sealing.

It is therefore highly desirable to provide an absorbent article that can be donned quickly, without embarrassing the user, and without exposing the user or the user's clothing to unsanitary conditions. To do so, the user must be able to quickly and easily don the absorbent article while partially disrobed in a confined space, such as a bathroom stall, while being assured that the absorbent article is properly aligned so that it will be comfortable and will not leak.

It is also desirable to provide an absorbent article that can be donned with one hand, which will facilitate quick and sanitary donning, and will allow use by individuals who may have a partial or complete inability to use two hands.

The present invention, as defined by the preferred embodiments, is designed to overcome the foregoing and other deficiencies of prior art absorbent garments.

### SUMMARY OF THE INVENTION

The features of the invention can be achieved by a disposable absorbent garment (“garment”) that can be fitted to the user without removing clothing from the user's legs. It is another feature of the invention to address the problems with prior art garments of this type. It is yet another feature of the invention to provide an inexpensive absorbent garment with a connector that can not be opened accidentally.

The garment generally comprises a front waist band, a rear waist band, and a crotch that may be manufactured as a single unit, as several combined units, or separately then joined together. Regardless of the construction used, the front and rear waist bands preferably are permanently joined to form a continuous loop, and the crotch preferably is permanently joined to the rear waist band and releasably connected to the front waist band.

In a demonstrative two-piece construction of the invention, the first part of the garment can be formed from a liquid impervious outer layer, a liquid pervious inner liner, and an absorbent layer disposed between the inner and outer layers. The inner, outer and absorbent layers have a rear waist region at one end and a crotch region at the other end. The crotch region is generally narrower than the rear waist region, such that together the crotch and rear waist regions form a “T” shape. Alternatively, the crotch region and the rear waist region can be approximately the same width.

The crotch region preferably has a front crotch edge located opposite the rear waist region, and a pair of laterally opposed inner leg-hole edges extending from the front waist edge towards the rear waist region. The inner leg-hole edges may have elastic gathers disposed in their vicinity. One or more gripping connectors preferably are disposed on the outer layer of the crotch portion near the front crotch edge. In one preferred embodiment, the gripping connectors and target connectors are of the hook and loop type, respectively.

The crotch region may be made with roughly parallel inner leg-hole edges to form a rectangular shape. Alternatively, the crotch region may be roughly hourglass shaped. In yet another alternative, the crotch region may be tapered at one end to provide it with a roughly trapezoidal shape.

The rear waist region has a top rear waist edge opposite the crotch region, a pair of laterally opposed rear side edges, and a rear outer leg-hole edge extending between each of the laterally opposed rear side edges and the inner leg-hole edges.

The second part of the garment is a front waist band. The front waist band has a top front waist edge opposite a front outer leg-hole edge, with laterally opposed front side edges extending therebetween. The second part of the garment is attached to the first part at the laterally opposed side edges of the front waist band and the rear waist region. Together, the front waist band and the rear waist region form a continuous waist band with a pair of side seams. The interior face of the front waist band has one or more target connectors disposed on it for joining the front of the crotch portion to the interior of the front waist band.

The front waist band and the rear waist region of the garment may be elasticized by one or more elastic elements, such as elastic films, strands or bands, which are attached to the waist band and waist region along all or part of their width. The elastic elements may have different properties and spacing that vary depending on the elastic's location in the waist band and/or waist region.

### BRIEF DESCRIPTION OF THE DRAWINGS

FIG. 1 is a partially cut-away view of an embodiment of the present invention, made with a two-piece construction, with the crotch fully-flattened and the waist band and waist regions separated;

FIG. 2 is a drawing of the two-piece embodiment of the present invention depicted in FIG. 1 with one seam connecting the front waist band to the rear waist region separated to more clearly show the garment;

FIG. 3 is a drawing of another two-piece embodiment of the present invention with one seam between the front waist band and the rear waist region separated to more clearly show the garment;

FIG. 4 is a drawing of a one-piece embodiment of the present invention with the seam between the front waist band and the rear waist region separated to more clearly show the garment;

FIG. 5 is a drawing of a three-piece embodiment of the present invention with the seam between the front waist band and the rear waist region separated to more clearly show the garment;

FIG. 6 is a drawing of a two-piece embodiment of the present invention with the seam between the front waist band and the rear waist region separated to more clearly show the garment;

FIG. 7 is a planar view of an embodiment of the present invention depicted in the fully-flattened position;

FIG. 8 is a planar view of another embodiment of the present invention depicted in the fully-flattened position; and,

FIG. 9 is a partially cut-away view of another embodiment of the present invention, made with a two-piece construction, with the crotch fully-flattened and the waist band and waist regions separated.

### DETAILED DESCRIPTION OF THE PREFERRED EMBODIMENTS

As used herein, the terms “absorbent garment,” “absorbent article” or simply “article” or “garment” refer to garments that absorb and contain exudates. More specifically, these terms refer to garments that are placed against or in proximity to the body of the wearer to absorb and contain the various exudates discharged from the body. A non-exhaustive list of examples of absorbent garments includes diapers, diaper covers, disposable diapers, training pants, feminine hygiene products and adult incontinence products. The term “disposable absorbent garment” refers to absorbent garments that are intended to be discarded or partially discarded after a single use (i.e., they are not intended to be laundered or otherwise restored or reused).

The absorbent garment of the preferred embodiments can be used with all of the foregoing classes of absorbent garments, without limitation, whether disposable or otherwise. These classifications are used interchangeably throughout the specification, but are not intended to limit the claimed invention. The invention will be understood to encompass, without limitation, all classes and types of absorbent garments, including those described above.

As used herein, the longitudinal axis of the garment is the dimension corresponding to the front-to-rear dimension of the user, and the lateral axis of the garment is the dimension corresponding to the side-to-side dimension of the user.

In general terms, the garment of an embodiment of the present invention is comprised of an elasticized waist band with a crotch component permanently attached to the back of the waist band and releasably attached to the front of the waist band. The crotch fits between the user's legs during use and the waist band wraps around the user's waist. The front of the waist band, rear of the waist band, and crotch may be manufactured as a single unit (“one-piece” construction), as various combinations of units (“two-piece” construction), or as three separate units (“three-piece” construction).

The garment further comprises an absorbent core, which is disposed in the crotch region and may also extend into the rear waist band region. The absorbent core is sandwiched between the topsheet and backsheet. At least part of the interior-facing topsheet surface of the garment is liquid-pervious to allow fluids to be absorbed into the absorbent core. The exterior-facing backsheet surface of the garment is generally liquid-impervious to prevent the fluid in the absorbent core from being released from the garment, but may be partly or wholly gas-pervious to provide the garment with breathability to enhance user comfort. An outer cover may also be placed on the liquid-impervious surface to improve the tactile feel or comfort of the garment.

These features, and other features, functions and uses of the present invention, are described in greater detail herein. For clarity, features that appear in more than one Figure have the same reference number in each Figure.

For simplicity, the preferred embodiments of the present invention will be described in terms of an adult incontinence garment. It should be understood, however, that the present invention is applicable to other types of absorbent garments.

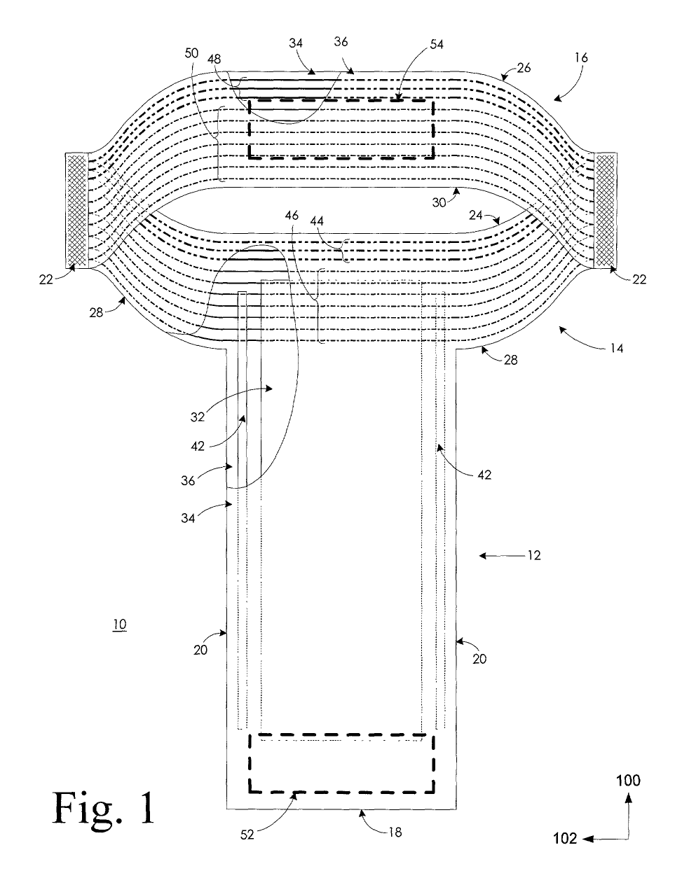

FIG. 1 is a partially cut-away view of an embodiment of the present invention with the crotch region fully-flattened and the waist parts separated for clarity. The garment 10 has a longitudinal dimension 100 and a lateral dimension 102. The garment 10 of FIG. 1 is made using a two-piece construction in which the crotch 12 and rear waist band 14 are made as one unit, and the front waist band 16 is made as a separate unit and then attached to the rest of the garment 10 at a pair of side seams 22.

The garment 10 is generally divided into a crotch 12, a rear waist band 14, and a front waist band 16. The crotch 12 extends between the wearer's legs during use. The crotch 12 is bounded by a laterally extending front crotch edge 18 and a pair of laterally opposite and longitudinally extending inner leg-hole edges 20. The inner leg-hole edges 20 are designed to contact the inner portion of each of the wearer's legs and form a seal to prevent leakage.

The crotch 12, rear waist band 14, and front waist band 16 are each comprised of multiple layers of material, which can be joined in a laminated construction. As used herein, the “topsheet” is the inner layer of any part of the garment 10 facing the wearer's body, and the “backsheet” is the outer layer of any part of the garment 10 facing away from the wearer's body.

An absorbent core 32 is disposed between the topsheet 34 and backsheet 36 in the crotch 12. If the crotch 12 and rear waist band 14 are made as a unit (i.e., sharing a common topsheet and a common backsheet), as is done in the embodiment depicted in FIG. 1, then the absorbent core 32 may also extend into the rear waist band 14. The absorbent core 32 may be made from any suitable material or materials known in the art. The invention is not intended to be limited to any specific materials of these components. In a preferred embodiment, the absorbent core 32 is comprised of super absorbent polymer distributed within a fibrous structure. Absorbent cores of this type are known in the art, and exemplary absorbent cores are described in U.S. Pat. No. 5,281,207, issued to Chmielewski et al., and U.S. Pat. No. 5,863,288, issued to Baker, which are herein incorporated by reference in their entirety.

The absorbent core 32 may also have one or more transfer layers or acquisition layers for assisting the core in handling fluid surges, preventing rewet, or other purposes. Those skilled in the art are capable of selecting materials, dimensions, and locations for such layers using the guidelines provided herein.

The topsheet 34 and backsheet 36 may be constructed from a wide variety of materials known in the art. The invention is not intended to be limited to any specific materials of these components. The topsheet 34 and backsheet 36 are shaped and sized according to the requirements of each of the various types of absorbent garment or to accommodate various user sizes.

The backsheet 36 is generally made of any suitable pliable liquid impervious material known in the art or later discovered. Typical backsheet materials include films of polyethylene, polypropylene, polyester, nylon, and polyvinyl chloride and blends of these materials. For example, the backsheet 36 can be made of a pigmented polyethylene film having a thickness in the range of 0.02-0.04 mm.

In addition, the backsheet 36 may be covered with a fibrous, nonwoven fabric such as is disclosed, for example, in U.S. Pat. No. 4,646,362 issued to Heran et al., which is hereby incorporated by reference units entirety and in a manner consistent with the present application and invention. Materials for such a fibrous outer liner include a spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers.

The backsheet 36 may further comprise separate regions having different properties. In a preferred embodiment, portions of the backsheet 36 are air-permeable to improve the breathability, and therefore comfort, of the garment 10. The different regions may be formed by making the backsheet 36 a composite of different sheet materials, chemical treatment, heat treatment, or other processes or methods known in the art. Some regions of the backsheet 36 may be fluid pervious. In one embodiment of the invention, the backsheet 36 is fluid impervious in the crotch 12, but is fluid pervious in the rear waist band 14 near the side seams 22, and the front waist band 16. The backsheet 36 may also be made from a laminate of overlaid sheets of material.

The moisture-pervious topsheet 34 can be made of any suitable relatively liquid-pervious material currently known in the art or later discovered that permits passage of a liquid therethrough. Non-woven materials are exemplary because such materials readily allow the passage of liquids to the underlying absorbent core 32. Examples of suitable topsheet materials include non-woven spun-bonded or carded webs of polypropylene, polyethylene, nylon, polyester and blends of these materials.

The topsheet 34 may also be made of single-ply nonwoven material that may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine (transverse) direction strength suitable for use as a topsheet material for the given application.

The topsheet 34 may further comprise several regions having different properties. In one embodiment of the present invention, the laterally distal portions of the topsheet 34 are preferably substantially fluid-impervious and hydrophobic. Different topsheet properties, such as fluid perviousness and hydrophobicity, may be imparted upon the topsheet 34 by treating the topsheet 34 with adhesives, surfactants, or other chemicals, using a composite of different materials, or by other means. The topsheet 34 may also be made from a laminate of overlaid sheets of material.

The backsheet 36 and the topsheet 34 are “associated” with one another. The term “associated” encompasses configurations whereby the topsheet 34 is directly joined to the backsheet 36 by affixing the topsheet 34 directly to the backsheet 36, and configurations whereby the topsheet 34 is indirectly joined to the backsheet 36 by affixing the topsheet 34 through intermediate members, which in turn are affixed to the backsheet 36. The topsheet 34 may be joined to the backsheet 36 by any of a number of means known in the art. Exemplary attachment means include use of lines of hot melt adhesive, ultrasonic bonding and the like. While the backsheet 36 and topsheet 34 in the preferred embodiment have substantially the same dimensions, they may also have different dimensions.

Referring still to FIG. 1, the crotch 12 may also include any of a number of mechanical sealing devices to provide the garment 10 with a leak-proof fit around the wearer. In a preferred embodiment, the inner leg-hole edges 20 have elastic gathers 42 placed along them to help the inner leg-hole edges 20 contract about the contours of the wearer's body, thereby providing a leak-proof seal. Such gathers 42 are well known in the art, and are disclosed, for example, in U.S. Pat. No. 5,830,203, issued to Suzuki et al., which is herein incorporated by reference in its entirety in a manner consistent with the present invention. The gathers 42 may extend into the rear waist band 14, and may extend as far as the front crotch edge 18.

In another preferred embodiment, standing leg gathers are disposed on the topsheet 34 in the crotch 12 above the absorbent core 32. Standing leg gathers are known in the art, and disclosed in U.S. Pat. No. 5,292,316, issued to Suzuki, which is herein incorporated by reference in its entirety in a manner consistent with the present invention. The standing leg gathers may extend into the rear waist region, and may extend as far as the front crotch edge 18.

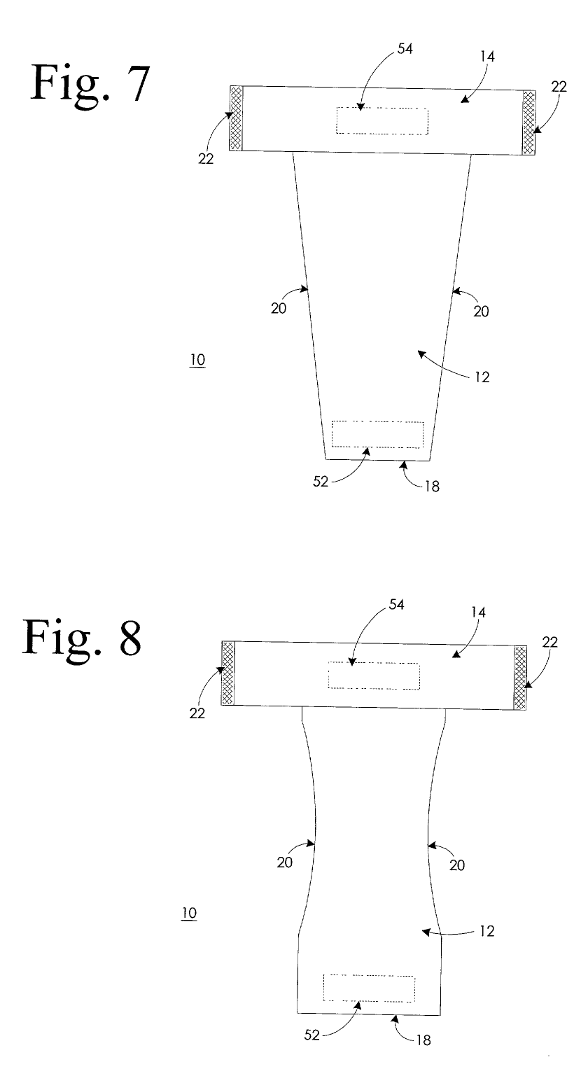

The inner leg-hole edges 20 may be made in a variety of shapes. In the preferred embodiment depicted in FIG. 1, the inner leg-hole edges 20 are substantially parallel and laterally opposed to one another. In another preferred embodiment, depicted in FIG. 7, the inner leg-hole edges 20 are closer to one another at the front crotch edge 18 than they are at the rear waist band 14, thereby forming a trapezoidal shape. In yet another preferred embodiment, depicted in FIG. 8, the inner leg-hole edges 20 are closer to one another together in the longitudinally central portion of the crotch 12 to provide the crotch 12 with an hourglass shape. Other shapes may also be employed to provide improved fit, comfort, leakage protection or manufacturing benefits using techniques known in the art.

Referring back to FIG. 1, the rear waist band 14 and front waist band 16 generally wrap around the wearer's waist during use in a belt-like manner and contain, respectively, laterally distal rear edges and laterally distal front edges (not shown). The respective laterally distal edges of the front waist band 16 and rear waist band 14 can be permanently joined at two laterally opposed side seams 22. The side seams 22 may be made by a variety of means well-known in the art, such as ultrasonic bonding, hot melt adhesive bonding, stitching, and the like. Although the side seams 22 are permanently bonded, they may be destructively torn apart to aid in removing the garment 10 from the wearer. Typically, if side seams 22 are destructively torn apart to aid in removing the garment 10, then can not be rejoined without the use of some external force. Alternatively, side seams 22 may be capable of repeated detachment and reattachment by use of, for example, a hook and loop type fastener. PATENT

The front and rear waist bands 16, 14 are elasticized so that they contract around the wearer's waist and dynamically fit the wearer as the wearer moves. The elasticized front and rear waist bands 16, 14 also seal the garment 10 against the wearer's skin to prevent leakage out of the top rear waist edge 24, top front waist edge 26, the rear outer leg-hole edges 28, and the front outer leg-hole edge 30. Together, the front and rear outer leg-hole edges 30, 28 and the inner leg-hole edges 20 form a continuous seal around the user's legs.

The front and rear waist bands 16, 14 may be elasticized by any of a variety of means known in the art. It is preferred that the front and rear waist bands 16, 14 have one or more elastic elements disposed between the topsheet 34 and backsheet 36. During a preferred construction process, the elastics are stretched before being sandwiched and affixed between the topsheet 34 and backsheet 36, so that when the elastics contract, the topsheet 34 and backsheet 36 that form each of the waist bands 14, 16 contract around the user's body. In another embodiment, an inelastic material that becomes elastic when exposed to heat can be sandwiched between the topsheet 34 and backsheet 36 in a relaxed state, and then heated to elasticize the waist bands. Such a material and process is disclosed in U.S. Pat. No. 4,640,859, issued to Hansen et al. The disclosure of which is incorporated herein by reference in its entirety.

The elastic element or elements may be made from an elastic film, such as that disclosed in U.S. Pat. No. 6,159,584, issued to Eaton et al., which is herein incorporated by reference in its entirety in a manner consistent with the present invention. The elastic element or elements may also be made from a multidirectional elastic aggregate such as elastic webbing, netting, or scrim elastic, such as FLEXCEL™ Elastic Nonwoven Laminate, available from Kimberly-Clark Corporation, headquartered in Neenah, Wis. The elastic element or elements may also be made from strands or bands of suitable elastic materials, such as rubber, spandex, LYCRA and elastic polymers.

In a preferred embodiment of the present invention, the front waist band 16 comprises a plurality of front waist elastics 48, preferably from about 1 to about 10 front waist elastics 48, and a plurality of front stomach elastics 50. The rear waist band preferably includes a plurality of rear waist elastics 44, preferably from about 1 to about 10 rear waist elastics 44, and a plurality of rear stomach elastics 46. The rear and front waist elastics 44, 48 preferably, are located near the top rear waist edge 24 and the top front waist edge 26, respectively. The rear and front stomach elastics 46, 50 preferably are located between the rear and front waist elastics 44, 48 and the rear and front outer leg-hole edges 28, 30, respectively. The waist elastics 44, 48 may have different stretch properties than the stomach elastics 46, 50 to help the garment 10 conform to the user's body contours. For example, the stomach elastics 46, 50 may resist extension of the front and rear waist bands 16, 14 less than the waist elastics 44, 48. With this construction, the lower portions of the waist bands, in which the stomach elastics are located will fit more comfortably around the user's hips and stomach, which are generally larger that the user's waist. Providing different stretch properties between the waist elastics 44, 48 and stomach elastics 46, 50 may be done by using materials with different spring constants, altering the spacing between each elastic, varying the cross-sectional dimensions of each elastic element, or by various other means known in the art. In addition, the rear waist and stomach elastics 44, 46 may have different properties than the front waist and stomach elastics 48, 50.

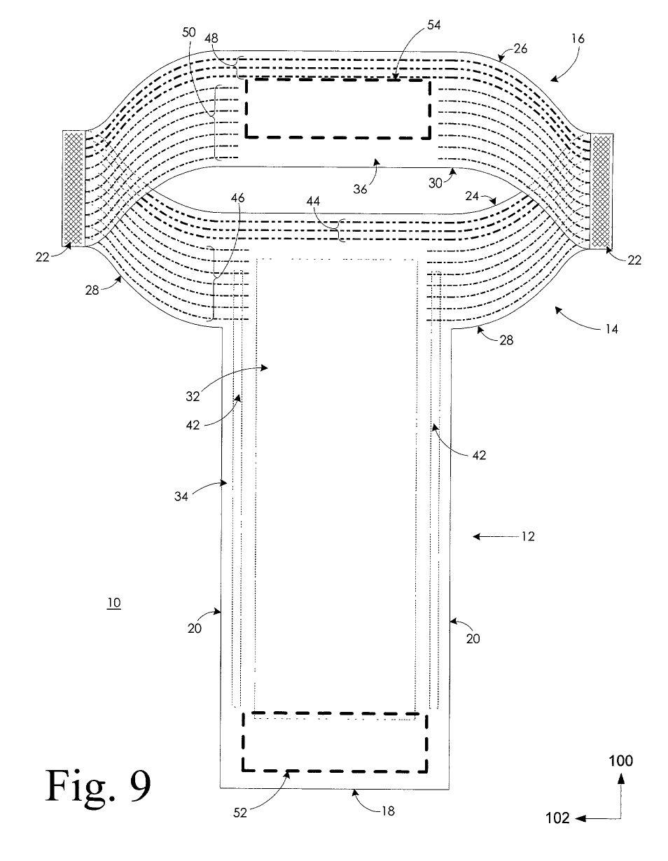

In the preferred embodiment depicted in FIG. 1, the elastics 44, 46, 48, 50 extend across the entire lateral dimension 102 of the waist bands 14, 16. In another preferred embodiment, one or more of the elastics 44, 46, 48, 50 are placed only across portions of the waist bands 14, 16. For example, FIG. 9 depicts another preferred embodiment in which the front and rear waist elastics 48, 44 extend across the entire lateral dimension 102 of the front and rear waist bands 16, 14, respectively, but the front and rear stomach elastics 50, 46 extend only across the laterally distal portions of the waist bands 14, 16, and not across the center regions of the waist bands.

Referring again to FIG. 1, a gripping connector 52 preferably is attached to the backsheet 36 near the front crotch edge 18. A target connector 54 preferably is attached to the front waist band 16 topsheet 34 between the front outer leg-hole edge 30 and the front top waist edge 26. The gripping and target connectors 52, 54 are designed to be releasably connected to one another, so that the garment 10 can be easily removed or adjusted after being donned. Any of a number of well known devices may be used for the gripping and target connectors 52, 54. In a preferred embodiment, the gripping and target connectors 52, 54 are hook and loop type fasteners, respectively.

The location of the gripping and target connectors 52, 54, provide additional assurance that the garment 10 will not accidentally open. The gripping and target connectors 52, 54 preferably are covered by the front waist band 16 while the garment 10 is being worn, and this prevents either connector from becoming snagged on or attached to the user's clothing.

Although the invention has thus far been described in terms of a two-piece construction with the crotch 12 and rear waist band 14 being made as a single unit and the front waist band 16 being made separately and then joined to the rear waist band/crotch unit, the present invention can be made with a variety of construction configurations.

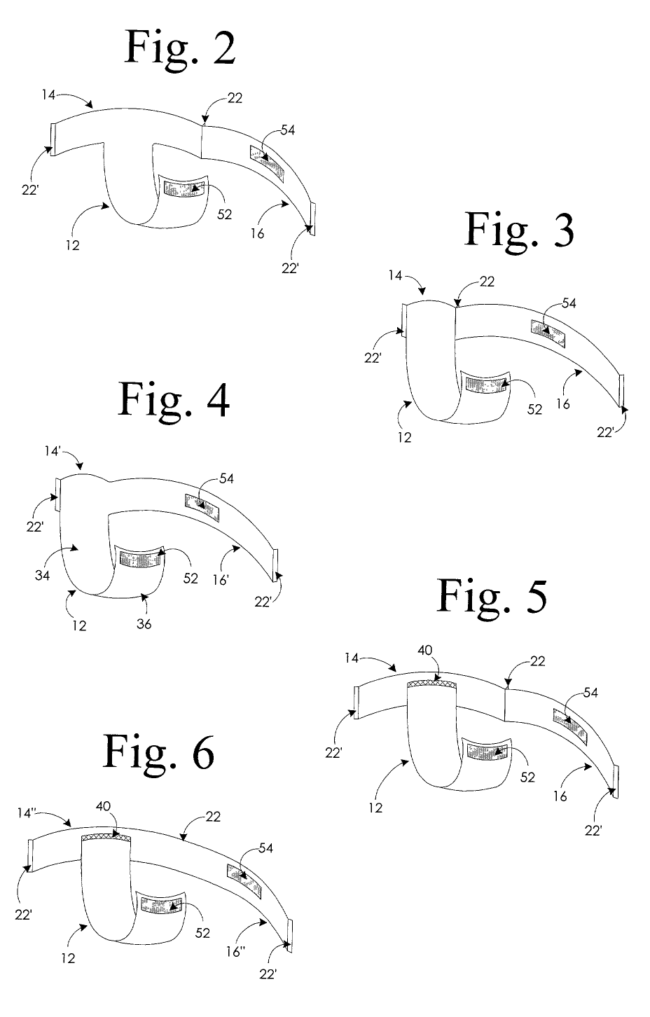

FIG. 1 depicts a preferred embodiment of the invention in which the rear waist band 14 and the crotch 12 are made together from continuous pieces of topsheet and backsheet material, and the front waist band 16 is made separately and joined to the rear waist band 14 along two side seams 22. FIG. 2 shows the embodiment depicted in FIG. 1 with one of the side seams 22′ connecting the front waist band 16 to the rear waist band 14 separated to more clearly show the structure of the garment 10. In this embodiment, the rear waist band 14 has a substantially greater lateral dimension 102 than the crotch 12. The gripping connector 52 and target connector 54 are visible in FIG. 2.

FIG. 3 depicts another preferred embodiment in which the garment 10 is made using a two-piece construction. The rear waist band 14 has approximately the same lateral dimension as the crotch 12, and the rear waist band 14 and crotch 12 are made together from continuous pieces of topsheet and backsheet material. Such a construction may provide substantial manufacturing benefits by, for example, reducing the number of cutting processes and reducing the amount of wasted material. Other manufacturing benefits may also be realized by this construction. Again, one side seam 22′ is shown separated for clarity, and the gripping connector 52 and target connector 54 are shown.

FIG. 4 depicts yet another embodiment of the present invention in which a one-piece construction is used. The crotch 12, rear waist band portion 14′, and front waist band portion 16″ are all constructed using the same topsheet 34 and backsheet 36. In such a configuration there is only one seam 22′, which is shown separated for clarity. Again, FIG. 4 shows the gripping connector 52 and target connector 54. This construction may provide additional manufacturing benefits, such as by reducing the number of joining operations.

In yet another preferred embodiment, depicted in FIG. 5, the crotch 12 and the rear waist band 14 are manufactured out of separate pieces of topsheet and backsheet material, then permanently bonded to each other along back seam 40. The back seam 40 can be made by any number of methods well known in the art, such as ultrasonic bonding, hot melt adhesives, and the like. FIG. 5 also depicts the embodiment with one seam 22′ separated for clarity, and shows the positions of the gripping connector 52 and target connector 54. Using this three piece construction, the above-mentioned benefits and other manufacturing benefits may be realized. For example, separate production of the crotch 12 and front and rear waist bands 16, 14 can reduce the number of non-machine direction processes that are required to manufacture the garment 10.

FIG. 6 depicts yet another preferred embodiment of the present invention which uses a two-piece construction. The crotch 12 is manufactured separately, and the waist band is comprised of continuous a rear waist band portion 14″ and front waist band portion 16″, which are manufactured together as a single unit. This construction includes a back seam 40 joining the crotch to the rear portion of the waist band 14″. The side seam 22′ again is shown separated for clarity, and the gripping connector 52 and target connector 54 are also shown in FIG. 6. Such a construction reduces the number of joining operations required, and may provide other additional manufacturing benefits.

Various other waist band and crotch construction configurations may be made under the present invention. Although FIGS. 2 through 6 depict the various embodiments of the present invention with one side seam 22′ separated, it is noted that this is done only for clarity. It is preferred in the invention that the side seams 22 are permanently attached during the manufacturing process.

The garment 10 may be donned more easily and with less exposure to unsanitary surfaces than traditional garments. For example, the garment 10 may donned by following these procedures:

pull the garment 10, with the crotch 12 unattached to the front waist band 16, up or down over the user's clothing until the front and rear waist band 16, 14 are around the user's waist;

partially remove the user's clothing;

remove any absorbent articles currently being worn;

while still holding the clothing with one hand, reach between the user's legs and pull the crotch 12 forward and tuck the front crotch edge 18 under the front outer leg-hole edge 30;

using one or both hands, reach under the front waist band 16 and adjust the gripping connector 52 and target connector 54 until the garment 10 is comfortable and tightly sealed on the user.

The above exemplary procedures make it possible to minimize the amount of time that the user must partly disrobe, which minimizes exposure to unsanitary surfaces. The above procedures also allow the user to use one hand to balance himself/herself or hold his/her clothing during the process. Furthermore, the above procedures for donning the garment can also be accomplished by a person who is able to use only one hand. The present invention may also be used in relatively public places, but will minimize the amount of embarrassment that the user may feel because the user is exposed for relatively little time. The location of the closure system also inhibits accidental openings because the connectors are separated from the user's clothing by the outer surface of the garment (e.g. front waist band 16).

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

We claim:

1. A disposable absorbent garment that can be fitted to the user without removing clothing from the user's legs comprising:
an outer layer comprised of a liquid impervious material;
a liquid pervious inner layer;
an absorbent layer disposed between the inner and outer layers, the inner, outer and absorbent layers having a rear waist region at one end and a crotch region at the other end,
the crotch region having a front crotch edge opposite the rear waist region, and a pair of laterally opposed inner leg-hole edges extending from the front crotch edge towards the rear waist region;
the rear waist region having a top rear waist edge opposite the crotch region, a pair of laterally distal rear edges, and rear outer leg-hole edges extending between each of the laterally distal rear edges and the inner leg-hole edges of the crotch region;
wherein the crotch region is generally narrower than the rear waist region;
one or more gripping connectors operably attached to the outer layer near the front crotch edge;
a front waist band having an interior face adjacent to the inner layer, a top front waist edge opposite a front outer leg-hole edge, with laterally distal front edges extending therebetween; said laterally distal front edges being operably attached to said laterally distal rear edges to form a pair of side seams;
one or more target connectors operably attached to the interior face of the front waist band, the target connectors adapted each to receive at least one of the one or more gripping connectors.

2. The garment of claim 1, wherein the rear waist region comprises a plurality of rear elastic elements operably attached to the rear waist region and extending at least partly between each of the laterally distal rear edges.

3. The garment of claim 2, wherein the plurality of rear elastic elements comprises:
from about one to about 10 rear waist elastics disposed near the top rear waist edge; and
a plurality of rear stomach elastics disposed between rear waist elastics and rear outer leg-hole edges.

4. The garment of claim 1, wherein the front waist band comprises a plurality of front elastic elements operably attached to the front waist band and extending at least partly between each of the laterally distal front side edges.

5. The garment of claim 4, wherein the plurality of front elastic elements comprises:
from about one to about 10 front waist elastics disposed near the top front waist edge; and
a plurality of front stomach elastics disposed between the front waist elastics and the front outer leg-hole edge.

6. The garment of claim 1, wherein the one or more gripping connectors and the one or more target connectors are hook-type fasteners and loop-type fasteners, respectively.

7. The garment of claim 1, further comprising elastic gathers disposed in each of the laterally opposed inner leg-hole edges in the crotch region.

8. The garment of claim 1, wherein the laterally opposed inner leg-hole edges are roughly parallel, and the crotch region has a roughly rectangular shape.

9. The garment of claim 1, wherein the laterally opposed inner leg-hole edges are closer to one another near the middle of the crotch region, and the crotch region has a roughly hourglass shape.

10. The garment of claim 1, wherein the laterally opposed inner leg-hole edges are closer to one another near the front crotch edge of the crotch region, and the crotch region has a roughly trapezoidal shape.

11. A disposable absorbent garment that can be fitted to the user without removing clothing from the user's legs comprising:
an outer layer comprised of a liquid impervious material;
a liquid pervious inner layer;
an absorbent layer disposed between the inner and outer layers, the inner, outer and absorbent layers having a rear waist region at one end and a crotch region at the other end,
the crotch region having a front crotch edge opposite the rear waist region, and a pair of laterally opposed inner leg-hole edges extending from the front crotch edge towards the rear waist region;
the rear waist region having a top rear waist edge opposite the crotch region, and a pair of laterally distal rear edges extending between the top rear waist edge and the pair of laterally opposed inner leg-hole edges;
the crotch region being approximately the same width as the rear waist region;
one or more gripping connectors operably attached to the outer layer near the front crotch edge;
a front waist band having an interior face adjacent to the inner layer, a top front waist edge opposite an outer leg-hole edge, with laterally distal front edges extending therebetween; the laterally opposed front side edges being operably attached to the laterally distal rear edges to form a pair of side seams; and
one or more target connectors operably attached to the interior face of the front waist band, the target connectors adapted each to receive at least one of the one or more gripping connectors.

12. The garment of claim 11, wherein the rear waist region comprises a plurality of rear elastic elements operably attached to the rear waist region and extending at least partly between each of the laterally distal rear edges.

13. The garment of claim 12, wherein the plurality of rear elastic elements comprises:
from about one to about 10 rear waist elastics disposed near the top rear waist edge; and
a plurality of rear stomach elastics disposed between the rear waist elastics and the crotch region.

14. The garment of claim 11, wherein the front waist band comprises a plurality of front elastic elements operably attached to the front waist band and extending at least partly between each of the laterally distal front edges.

15. The garment of claim 14, wherein the plurality of waist elastic elements comprises:
from about one to about 10 front waist elastics disposed near the top front waist edge; and
a plurality of front stomach elastics disposed between the front waist elastics and the outer leg-hole edge.

16. The garment of claim 11, wherein the one or more gripping connectors and the one or more target connectors are hook-type fasteners and loop-type fasteners, respectively.

17. The garment of claim 11, further comprising elastic gathers disposed in each of the laterally distal inner leg-hole edges in the crotch region.

18. The garment of claim 11, wherein the laterally distal inner leg-hole edges are roughly parallel, and the crotch region is roughly rectangular-shape.

19. The garment of claim 11, wherein the laterally distal inner leg-hole edges are closer to one another near the middle of the crotch region, and the crotch region has a roughly hourglass shape.

20. The garment of claim 11, wherein the laterally distal inner leg-hole edges are closer to one another near the front crotch edge of the crotch region, and the crotch region has a roughly trapezoidal shape.

21. A disposable absorbent garment that can be fitted to the user without removing clothing from the user's legs comprising:
an outer layer comprised of a liquid impervious material;
a liquid pervious inner layer;
an absorbent layer disposed between the inner and outer layers, the inner, outer and absorbent layers having a waist region at one end and a crotch region at the other end,
the crotch region having a front crotch edge opposite the waist region, and a pair of laterally distal inner leg-hole edges extending from the front crotch edge towards the waist region;
the waist region having a top waist edge opposite the crotch region, a pair of laterally distal edges, and at least one outer leg-hole edge extending between at least one of the laterally distal edges and at least one of the laterally distal inner leg-hole edges;
the laterally distal edges being operably attached to one another to form a side seam wherein the waist region forms a continuous waist band having an interior surface;
the crotch region being substantially narrower than the waist region;
one or more gripping connectors operably attached to the outer layer near the front crotch edge;
one or more target connectors, operably attached to the interior surface of the waist band, the target connectors adapted each to receive at least one of the one or more gripping connectors,
wherein the target connectors are oriented such that when the gripping connectors are attached to the target connectors the laterally distal inner leg-hole edges and the outer leg hole edge form a pair of leg-holes.

22. The garment of claim 21, wherein the rear waist region comprises a plurality of elastic elements operably attached to the waist region and extending at least partly between each of the laterally distal edges.

23. The garment of claim 22, wherein the plurality of elastic elements comprises:
from about one to about 10 waist elastics disposed near the top waist edge; and
a plurality of stomach elastics disposed between the waist elastics and the crotch region.

24. The garment of claim 21, wherein the one or more gripping connectors and the one or more target connectors are hook-type fasteners and loop-type fasteners, respectively.

25. The garment of claim 21, further comprising elastic gathers disposed in each of the laterally distal inner leg-hole edges in the crotch region.

26. The garment of claim 21, wherein the laterally distal inner leg-hole edges are roughly parallel, and the crotch region is roughly rectangular shape.

27. The garment of claim 21, wherein the laterally distal inner leg-hole edges are closer to one another near the middle of the crotch region, and the crotch region has a roughly hourglass shape.

28. The garment of claim 21, wherein the laterally distal inner leg-hole edges are closer to one another near the front crotch edge of the crotch region, and the crotch region has a roughly trapezoidal shape.

29. A disposable absorbent garment that can be fitted to the user without removing clothing from the user's legs comprising:
a rear waist band having a top rear waist edge opposite a rear outer leg-hole edge, and laterally distal rear edges extending therebetween;
a front waist band having an inner face adjacent the rear waist band, a top front waist edge opposite a front outer leg-hole edge and laterally distal front edges extending therebetween; the laterally distal front side edges being operably attached to the laterally distal rear side edges to form a pair of side seams;
a crotch portion comprising an outer layer comprised of a liquid impervious material, a liquid pervious inner layer, and an absorbent layer disposed between the inner and outer layers, the inner, outer and absorbent layers having a rear crotch edge at one end and a front crotch edge at the opposite end, with a pair of inner leg-hole edges therebetween,
the rear crotch edge being operably attached to the rear waist band;
one or more gripping connectors operably attached to the outer layer near the front crotch edge; and
one or more target connectors operably attached to the inner face of the front waist band, the target connectors adapted each to receive at least one of the one or more gripping connectors.

30. The garment of claim 29, wherein the rear waist band comprises a plurality of rear elastic elements operably attached to the rear waist region and extending at least partly between each of the laterally distal rear edges.

31. The garment of claim 30, wherein the plurality of rear elastic elements comprises:
from about one to about 10 rear waist elastics disposed near the top rear waist edge; and
a plurality of rear stomach elastics disposed between the rear waist elastics and the rear outer leg-hole edge.

32. The garment of claim 29, wherein the front waist band comprises a plurality of front elastic elements operably attached to the front waist band and extending at least partly between each of the laterally distal front edges

33. The garment of claim 32, wherein the plurality of front elastic elements comprises:
from about one to about 10 front waist elastics disposed near the top front waist edge; and
a plurality of front stomach elastics disposed between the front waist elastics and the front outer leg-hole edge.

34. The garment of claim 29, wherein the one or more gripping connectors and the one or more target connectors are hook-type fasteners and loop-type fasteners, respectively.

35. The garment of claim 29, further comprising elastic gathers disposed in each of the laterally distal inner leg-hole edges in the crotch region.

36. The garment of claim 29, wherein the laterally distal inner leg-hole edges are roughly parallel, and the crotch region has a roughly rectangular shape.

37. The garment of claim 29, wherein the laterally distal inner leg-hole edges are closer to one another near the middle of the crotch region, and the crotch region has a roughly hourglass-shaped.

38. The garment of claim 29, wherein the laterally distal inner leg-hole edges are closer to one another near the front crotch edge of the crotch region, and the crotch region has a roughly trapezoidal shape.

39. A disposable absorbent garment that can be fitted to the user without removing clothing from the user's legs comprising:
a waist band having a top waist edge opposite an outer leg-hole edge, and laterally distal edges extending therebetween, the laterally distal edges being operably attached to one another such that the waist band forms a continuous waist band with an interior surface;
a crotch portion comprising an outer layer comprised of a liquid impervious material, a liquid pervious inner layer, and an absorbent layer disposed inner and outer layers, the inner, outer and absorbent layers having a rear crotch edge at one end and a front crotch edge at the opposite end, with a pair of inner leg-hole edges therebetween, the rear crotch edge being operably attached to the continuous waist band;
one or more gripping connectors operably attached to the outer layer near the front crotch edge;
one or more target connectors operably attached to the interior surface of the waist band, the target connectors adapted each to receive at least one of the one or more gripping connectors;
the target connectors being located such that when the gripping connectors are attached to the target connectors the laterally distal inner leg-hole edges and the outer leg hole edge form a pair of leg-holes.

40. The garment of claim 39, wherein the waist band comprises a plurality of rear elastic elements operably attached to the rear waist band and extending at least partly between each of the laterally distal edges.

41. The garment of claim 40, wherein the plurality of elastic elements comprises:
from about one to about 10 waist elastics disposed near the top waist edge; and
a plurality of stomach elastics disposed between the waist elastics and the outer leg-hole edge.

42. The garment of claim 39, wherein the one or more gripping connectors and the one or more target connectors are hook-type fasteners and loop-type fasteners, respectively.

43. The garment of claim 39, further comprising elastic gathers disposed in each of the laterally distal inner leg-hole edges in the crotch region.

44. The garment of claim 39, wherein the laterally distal inner leg-hole edges are roughly parallel, and the crotch region has a roughly rectangular shape.

45. The garment of claim 39, wherein the laterally distal inner leg-hole edges are closer to one another near the middle of the crotch region, and the crotch region has a roughly hourglass shape.

46. The garment of claim 39, wherein the laterally distal inner leg-hole edges are closer to one another near the front crotch edge of the crotch region, and the crotch region has a roughly trapezoidal shape.
